(19) United States
(12) Patent Application Publication
(10) Pub. No.: US 2025/0248968 A1
(43) Pub. Date: Aug. 7, 2025

(54) COMPOSITION COMPRISING CAPSAICIN OR A CAPSACINOID

(71) Applicant: MESTEX AG, Basel (CH)

(72) Inventors: Christian GERBER, Zug (CH); Thomas GILLER, Wintersingen (CH); Olivier VALDENAIRE, Basel (CH)

(73) Assignee: MESTEX AG, Basel (CH)

(21) Appl. No.: 19/184,734

(22) Filed: Apr. 21, 2025

Related U.S. Application Data
(63) Continuation of application No. 17/486,065, filed on Sep. 27, 2021.
Application No. 17/486,065 is a continuation of application No. PCT/CH2019/000008, filed on Mar. 28, 2019.

(51) Int. Cl. A61K 31/36; A61K 9/00; A61K 31/165; A61K 31/167; A61K 31/445; A61P 29/00; A61P 41/00

(52) U.S. Cl. CPC A61K 31/36; A61K 9/0019; A61K 31/165; A61K 31/167; A61K 31/445; A61P 29/00; A61P 41/00

(57) ABSTRACT

A composition comprising capsaicin or a capsacinoid is used in a method for postoperative pain control. The composition is administered to a site intended for surgery in a patient at least one day before surgery is actually performed.

This application is a continuation of U.S. Nonprovisional application Ser. No. 17/486,065, filed Sep. 27, 2021, now pending, which is, in turn, a continuation of International Patent Application No. PCT/CH2019/000008, filed Mar. 28, 2019, the entire contents of which patent applications are hereby incorporated herein by reference.

The present invention generally relates to a composition comprising capsaicin or a capsacinoid for use in a method for postoperative pain control.

Post-operative pain is one of the most important reasons for a prolonged hospital stay after surgery. Furthermore, suffering for the patients can be relevant and maybe one of the main reasons for a delayed rehabilitation. Therefore numerous attempts have been made to treat post-operative pain with appropriate medication. This includes, among various forms of local, regional or systemic administration, oral, intravenous, topical, rectal applications or other ways of any type of know pain medication including non-steroidal or anti-inflammatory drugs (NSAID), Paracetamol, opioids and similar drugs such as for example local anesthetics or similar drugs. These drugs are usually given during the hospital stay and directly during the operation and in the period afterwards. However, all these drugs and treatments have incomplete effectiveness, show significant side effects or even lose efficacy over time.

In particular new approaches have been tried to control pain after joint surgery, which have also been used after visceral surgery and other type of surgical intervention. For example, a bupivacaine (local analgesic) liposome injectable suspension was administered at the time of surgery to control pain and reduce or eliminate the use of opioids for acute postsurgical pain. However, the effect of this medication is relatively short timed, lasting only a few days and is also often incomplete. Another approach was to treat the surgical site during or at the end of an operation with capsacinoids to eliminate postoperative pain. Unfortunately this approach has not proven to be reliably effective.

Thus, there remains a need for a composition comprising capsaicin or a capsacinoid for use in an improved method for postoperative pain control overcoming the above mentioned disadvantages.

The composition according to the invention has shown to surprisingly well address all kinds of postoperative pain, being effective for most of or the entire time of rehabilitation.

The principle is, to inject a formulation of capsaicin or a capsacinoid such as resiniferatoxin (RTX) at least one day, preferably one or two weeks, ideally one month before the operation into the surgical field. In extreme cases the method is also effective when applied several months up to one year before surgery.

Repeated injections preoperatively for example two and six weeks preoperatively may achieve the same or better effect, with the advantage being that the effective dose given can be increased with each infiltration due to increased tolerance for the injected capsaicin or capsacinoid regarding pain after infiltration. In the example of a joint operation, the injection will take place into the synovial cavity, but may also be injected around the joint, which may also be effective but is usually technically more demanding and less comfortable for the patient. Surprisingly, this injection will prepare the joint in such way, that postoperatively very little pain arises even though that procedure is performed far in advance of the surgery. Surprisingly, the desired effect is better and more consistent if there is a relevant time between injection and operation. The ideal time appears to be between two and six weeks preoperatively.

### INDICATIONS

Indications for such preventive pain treatment are in principle any surgical interventions, which are associated with all kind of postoperative pain. Particularly attractive and effective is the method to treat postoperative pain of orthopedic interventions and surgeries related to sports injuries, either from open, minimally invasive or arthroscopic surgery. These interventions may include joint replacement surgery, arthroplasty, debridement, cartilage repair, osteosynthesis, fracture treatment, treatment of ligament or tendon rupture or distortion (sprain), amputation, tumor or cancer surgery, in any joint, shoulder, elbow, hand, hip, knee, ankle or foot. In particular preoperative treatment is included for operations on the tennis elbow (medial or lateral epicondylitis) heel spur, hallux valgus, Haglund exostosis, jumpers knee, runners knee, patella pain syndrome, Mortons neuroma, hip impingement, iliofemoral or greater trochanteric bursitis or tendon pain, shoulder (subacromial or other) impingement, rotator cuff injury, calcifying tendonitis, biceps tendonitis or injury, rhizarthrosis, carpal tunnel syndrome, iliosacral joint, symphysis pain, intervertebral (facet) joints, intervertebral disc injuries or problems. Open or arthroscopic surgery such as tendon repair, cruciate ligament repair or reconstruction, meniscal surgery, cartilage repair, shoulder instability surgery, surgical treatment of joint stiffness in the shoulder, knee or any other joint. In the extremities or the spine open, mini-open, arthroscopic or endoscopic surgeries may include debridement, cartilage repair, removal of osteophytes or loose bodies, osteosynthesis, fracture treatment, treatment of ligament or tendon rupture, amputation, tumor or cancer surgery, bursectomy, infection treatment, spinal fusion, transplantations, nerve repair or transplantation, skin repair and transplantation at the donor or graft site. In dental or maxillofacial surgery, treatment may address any operation involving teeth (including tooth extraction), the mandibular joint, and nerve operations such as for the trigeminal nerve, reconstructive or aesthetic procedures, operations involving the nose such as plastic surgery, turbinoplasty and septum plasty. Further applications apply to visceral surgery, addressing the bowel (appendix), liver and gall bladder, urine bladder and reproductive organs. In thoracic surgery particularly interventions addressing the sternum (sternotomy), rib cage and pleura are included. The preventive injection procedure against postoperative pain is not limited to the above-described procedures and has also been effective in revision operations of the here mentioned operations. Problems arising in above-mentioned organs may be due to injury, overuse, natural ageing, inflammation, rheumatoid disease, osteoarthritis, tumor disease, infection and postoperative iatrogenic problems.

The here described methods of preoperative application of capsacinoids (RTX) has shown particularly promising effects to avoid postoperative algodystrophie syndroms such as morbus Sudeck, complex regional pain syndrome (CRPS), frozen shoulder, arthrofibrosis and similar conditions.

### LIST OF ACTIVE SUBSTANCES

Alternatively, to resiniferatoxin (RTX) capsacinoids may be used and are from the group of vanilloid receptor agonists, mainly acting on the TRPV1 channel, which is predominantly found on C-fibers and acts as a nerve signal trigger for pain or heat. These substances include mainly capsaicin and its analogues, pseudocapsaicin, dihydrocapsaicin, homocapsaicin, homodihydrocapsaicin, transcapsaicin, anandamide, civamide, nonivamide, olvanil, N-oleyl-homovanillamidia, isovelleral, scalaradial, ancistrodial, merulidial, scutigeral and any combinations or mixtures thereof.

### DOSAGES OF CAPSAICIN AND CAPSACINOIDS

Typical dosages of the active substances or mixtures thereof correspond to the equipotent dosage as given below: RTX 20-5000 ng, preferably 100-1000 ng, capsaicin 100microgramm to 10 miligramms, preferably 500-200 0microgramms.

### ADDITIONAL DOSAGE OF A LOCAL ANESTHETIC

Local anesthetics are used and known to alleviate the pain resulting from the injection of a capsacinoid. They are usually given immediately before or together or immediately after the administration of a capsacinoid (within minutes or hours before or after the capsacinoids). However, we made the unexpected discovery, that the previous or simultaneous application of local anesthetics together with capsacinoids is enforcing the efficacy of capsacinoids, in particular of RTX to reduce postoperative pain from a surgical site when administered there at a different time point before the surgery. The efficacy of RTX to reduce postoperative pain is also dependent on this minimal time frame and has been found to increase starting at few days preoperatively, reaching a maximum at 2 to 6 weeks preoperatively.

Doses of local anesthetics needed to increase the efficacy of RTX by 50% correspond approximately to the amount needed (details given below) to reduce the pain level of RTX injection by 50%. to 100%.

Doses of local anesthetics typically are solutions of 0.5 to 100 ml, preferably 1 to 30 ml of Lidocaine 0.5 to 5%, preferably 1 to 2%, Ropivacaine 0.1 to 5%, preferably 0.25 to 2%, Bupivacaine 0.1 to 5%, preferably 0.25 to 2%, Tetracaine 0.1 to 5%, preferably 1 to 2%, further Prilocain, Etidocaine, Procaine 0.1 to 5%, preferably 0.25 to 2%,

Mepivacain and Levobupivacaine 0.5 to 5%, preferably 1 to 2% or similar substances.

### BUFFER FORMATION

The agent may be in a carrier, a pharmacologically acceptable vehicle, in particular from the group of sodium chloride solution for injection, Ringer's solution for injection, isotonic dextrose, sterile water dextrose solution, Lactated Ringers injection solution, distilled water or mixtures thereof be resolved for local injection.

Preparation of the final injection solution includes the resuspension of RTX with a physiological buffer solution containing different salts (sodium, potassium, calcium, magnesium, chloride, ammonium or sulfate) to establish a physiological environment. To reach a suitable osmolarity the salts have to be in the following ranges: Natrium between 0 and 200 mM, Potassium, Magnesium and Calcium between 0 and 10 mM, Chloride between 0 and 500 mM, ammonium between 0 and 50 mM, sulfate between 0 and 200 mM. Furthermore, a pH buffering activity such as HEPES, phosphate, TRIS, Histidine, MOPS is required to establish the pH between 5.0 and 9.0 preferably between 7.5 and 8.0.

In another embodiment, in addition to RTX, calcium Ca ²⁺ or comparable ions in a concentration higher than physiologically present used in the solvent and released simultaneously or with a delay. Calcium is necessary for the action of RTX and enhances its effect when present in hyper-physiological concentration. The concentration of calcium is preferably >2 mmol, in particular >4 mmol. Some compounds or ingredients have also been shown to enhance the performance of RTX, e.g. Magnesium, Antioxidants, Preservatives and Excipients, especially sodium bisulfite (>0.2%), NaHSO₃, ammonium compounds, such as ammonium sulfate (NH₄) 2SO₄, 2-10 (−30%), Polysorbate 80 (PS80) 0.01 to 0.2 mg/ml, preferably 0.025 to 0.08 mg/ml.

The salts and ions dissolved in the dissolution medium are preferably concentrated higher than physiologically normal (e.g., in Ringer's lactate solution).

RTX is preferably dissolved in a biocompatible solvent and is conveniently injected in an amount that corresponds to the available space in the joint to be treated so that it fills up easily to bulging. This achieves the advantage of an optimal local distribution of RTX. But it is also possible to inject less fluid, but then the joint must be well moved for better distribution of the substance combination.

The solving agent may additionally contain a permeation enhancer, preferably dimethyl sulfoxide, ethoxyethylene diglycol, ethanol, phosphatidylcholines, propylene glycol dipelargonate (DPPG), or glycosylated glycerides.

Solubility enhancing and stabilizing agents may be used from the group of benzyl alcohols, butylated hydroxatoluenes, cremophores (EL, RH60), polyoxyethylene, sorbitanmonooleate or mannose to improve the activity of RTX.

The volume of liquid to be injected into the intracapsular area may be from 0.1 to 150 ml. For a finger joint about max. 1ml, for the shoulder joint max. 10 ml, for the knee joint max. about 30-50 ml, but preferably not over 20ml.

### LIST OF APPLICATION METHODS

A possible local anesthesia before, simultaneously with or after administration of RTX may be administered directly into the area of the surgical approach (skin incision, tissue preparation area) to the region of surgery, in the context of joint surgery around and or into (intraarticularly, into the synovial space) the joint space, to the sensory nerves connected to the area of surgery or as a regional or spinal anesthesia. The site of infiltration may be defined by controlling of the needle position or the distribution of a contrast medium mixed with local anesthetics or RTX or being administered prior to this by means of fluoroscopy, radiographs, computer tomography or ultrasound. If the position of the injection needle or any suitable alternative administration tool (small catheter, tube) has been verified, it may be left in place to ensure the identical site of injection for a local anesthetic and the RTX solution.

Possible sites for injection of local anesthetics and/or (local anesthesia is not mandatory) RTX include in principle any site of intended surgery within the body of a human or animal. Specifically the periarticular area (around the joint capsule) or intraarticular space (synovial space) of all big or small joints of the body such as shoulder, elbow, hand, fingers, hip, knee, foot, ankle, toes and intervertebral joints and discs (nucleus pulposus) are included.

Further any site of a tendon or ligament, particularly their insertion areas is included, such as for example the patellar tendon, the rotator cuff, the biceps tendon, triceps tendon, Achilles tendon, wrist extensors and flexors and plantar fascia, collateral ligament of elbow and knee.

Further advantageous embodiments of the invention can be commented as follows:

In a special embodiment of the invention the administering of the composition occurs at least 1 week, preferably at least 2 weeks before surgery. The administering of the composition can also occur at least 1 month, preferably at least 2 months before surgery.

In a further embodiment of the invention the administering of the composition is repeated several times preoperatively, preferably 2 to 6 weeks before surgery.

The capsacinoid can be selected from the group consisting of resiniferatoxin, N-vanillylnonanamides, N-vanillylsulfonamides, N-vanillylureas, N-vanillylcarbamates, N[(substituted phenyl)methyl]alkylamides, methylene substituted N[(substituted phenyl)methyl]alkanamides, N[(substituted phenyl)methyl]-cis-monosaturated alkenamides, N[(substituted phenyl)methyl]diunsaturated amides, 3-hydroxyacetanilide, hydroxyphenylacetamides, pseudocapsaicin,, homocapsaicin, homodihydrocapsaicin, transcapsaicin,, civamide, nonivamide, olvanil, N-oleyl-homovanillamidia,
dihydrocapsaicin, nordihydrocapsaicin anandamide, piperine, zingerone, warburganal, polygodial, aframodial, cinnamodial, cinnamosmolide, cinnamolide, isovelleral, scalaradial, ancistrodial, [beta]-acaridial, merulidial, scutigeral, and any combinations thereof.

Purposefully a dose of 500-2000 micrograms of capsaicin is administered. Preferably a dose of 20-5000 ng, more preferably of 100-1000 ng of resiniferatoxin is administered.

In a further embodiment, additionally to the pre-operative administering of capsaicin or a capsacinoid, a dose of local anesthetic is administered prior to, simultaneously with or posteriorly to the capsaicin or a capsacinoid administration. The local anesthetic is preferably administered 1 to 15 minutes prior to the capsaicin or capsacinoid administration.

Purposefully the local anesthetic is administered as a solution of 0.5 to 100 ml, preferably 1 to 30 ml of the local anesthetic. The concentration of the local anesthetic in the solution depending on the type of local anesthetic may be as follows:
for Lidocain 0.5 to 5%, preferably 1 to 2%;
for ropivacaine 0.1 to 5%, preferably 0.25 to 2%;
for bupivacaine 0.1 to 5%, preferably 0.25 to 2%;
for tetracaine 0.1 to 5%, preferably 1 to 2%;
for prilocain, etidocaine or procaine 0.1 to 5%, preferably 0.25 to 2%;
for mepivacain or levobupivacaine 0.5 to 5%, preferably 1 to 2%.

In a further embodiment the capsaicin or capsacinoid is dissolved in a carrier selected from the groups of a pharmacologically acceptable vehicle, in particular from the group of sodium chloride solution for injection, Ringer's solution for injection, isotonic dextrose, sterile water dextrose solution, Lactated Ringers injection solution, distilled water or mixtures thereof.

### EXAMPLE 1

A 62-year-old male patient with hip osteoarthritis and pain for 2 years was not treatable anymore with conventional pain medication (NSAIDS, Paracetamol, opioids, etc.) or infiltration (corticosteroids, hyaluronic acid). Therefore the patient was treated with an infiltration into the hip joint under fluoroscopic control with 0.8 micrograms of Resiniferatoxin dissolved in 250 microliters of Ethanol and mixed with 5 ml buffer solution.

10 minutes prior to the injection a local anesthetic (5 ml lidocaine 2%) was applied under fluoroscopic control intra-articularly to prevent injection pain. Moderate post-injection pain after RTX was controlled with oral administration of morphine. One month later the patient still suffered from relevant impairment of hip mobility and peritrochanteric pain and was treated with implantation of a total hip prosthesis. Surprisingly the patient reported moderate to none postoperative pain (VAS 5 on the first and VAS 2 on the second postoperative day). Thereafter virtually no pain was reported and the patient did not need any further per oral pain medication.

### EXAMPLE 2

A 35-year-old physiotherapist suffered a traumatic anterior cruciate ligament rupture with medial meniscal tear. Due to unacceptable symptoms of instability a surgical reconstruction was scheduled. During the waiting period for the operation 3 weeks preoperatively an intra-articular treatment with 2 micrograms of resiniferatoxin dissolved in Ringers lactate buffer at a concentration of 200 ng/ml with immediate prior administration (i.e. within 1 minute) of a local anesthetic (5 ml 0.5% bupivacaine) was applied.

Three weeks later a crucial ligament repair using patellar tendon autograft and arthroscopic meniscal repair was performed. Postoperative pain was restricted to the first 72 hours and below VAS 2 thereafter. Unexpectedly the passive range of motion was flex-extension of 130-0-0° 3 weeks after the operation and further improving thereafter with minimal (VAS 1) pain.

### EXAMPLE 3

A series of 5 patients with a mean age 62 years and transtendinous rotator cuff tears and having been scheduled for arthroscopic cuff repair was 2 to 6 weeks (13 days for two patients and 16,23 and 41 days for the other three patients) preoperatively treated with a subacromial injection of 0.4 microgram resiniferatoxin dissolved in 10 ml 2% lidocaine.

Postinjection pain was controlled with ice packs and NSAIDS. The postoperative pain protocol included 48 hours of interscalene continuous regional anesthesia. Thereafter pain was exceptionally low (VAS <2) in four out of five patients. Six weeks postoperatively none of the patients reported pain upon passive external rotation, which was in all patients within 15° of the contralateral side and reached at least 80° of glenohumeral abduction. Also within the further postoperative course up to 1 year follow up no clinical signs corresponding to a stiff or frozen shoulder were observed.

### EXAMPLE 4

A series of 3 patients with symptoms of frozen shoulders (VAS >6) for at least six month were scheduled for arthroscopic capsular release. All patients were treated with an intra-articular treatment with 2 micrograms of resiniferatoxin dissolved in 10 ml of physiological salt solution containing 4 mM Ca²⁺ with immediate prior administration (2.5 and 15 minutes prior to resiniferatoxin) of a local anesthetic (5 ml 0.25% ropivacaine) at 29, 32 and 40 days preoperatively.

Thereafter pain regressed 2 weeks after injection to a level below VAS 3 in all patients. The postoperative pain treatment after capsulotomy included 5 days of regional continuous anesthesia. After that period all of the patients reported pain on a level of VAS <3 and were able to fully maintain their intraoperatively achieved mobility.

### EXAMPLE 5

A patient with symptomatic hallux valgus was scheduled for surgical treatment. One month preoperatively an intra articular injection 1 ml resiniferatoxin (200 ng) was administered into the interphalangeal join space with prior (5 minutes) periarticular anesthesia using 2 ml 2% Lidocaine. Postoperative pain level was such low that the patient did not need further pain medication after the second day after surgery.

### EXAMPLE 6

A 28-year-old male patient with painful rizarthrosis was scheduled for surgical treatment of the disease on week prior to the operation. the patient was injected with 1 ml of a physiological saline solution containing 0.9 mg capsaicin into the painful joint base without prior administration of local anesthetics. The massive pain was treated with ice packs and inter-muscular morphine. However, following the surgical procedure almost no postoperative pain occurred.

Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims.

It is appreciated that certain features of the invention, which are, for clarity, described in the context of separate embodiments, may also be provided in combination in a single embodiment. Conversely, various features of the invention, which are, for brevity. described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

1. A method for postoperative control of pain resulting from surgery performed on a joint in a patient in need thereof, said method comprising administering to said patient at a site of said joint an effective amount of resiniferatoxin several times 2 to 6 weeks before said surgery is performed.

2. Method according to claim 1, wherein the administering of the composition occurs at least 1 month before surgery.

3. Method according to claim 1, wherein the administering of the composition is repeated several times preoperatively.

4. Method according to claim 1, wherein a dose of 20-5000 ng of resiniferatoxin is administered.

5. Method according to claim 1, wherein a dose of local anesthetic is administered prior to, simultaneously with or posteriorly to the resiniferatoxin.

6. Method according to claim 5, wherein the local anesthetic is administered 1 to 15 minutes prior to the resiniferatoxin.

7. Method according to claim 5, wherein the local anesthetic is administered as a solution of 0.5 to 100 ml of the local anesthetic.

8. Method according to claim 7, wherein the local anesthetic and a concentration of the local anesthetic in the solution is as follows:
for Lidocain 0.5 to 5%;
for ropivacaine 0.1 to 5%;
for bupivacaine 0.1 to 5%;
for tetracaine 0.1 to 5%;
for prilocain, etidocaine or procaine 0.1 to 5%; and
for mepivacain or levobupivacaine 0.5 to 5%.

9. Method according to claim 1, wherein the resiniferatoxin is dissolved in a carrier selected from the group consisting of sodium chloride solution for injection, Ringer's solution for injection, isotonic dextrose, sterile water dextrose solution, Lactated Ringers injection solution, distilled water, and mixtures thereof.
